(19) United States
(12) Patent Application Publication
(10) Pub. No.: US 2022/0053994 A1
(43) Pub. Date: Feb. 24, 2022

(54) METHODS AND FORMULATIONS FOR ATHLETIC SHOE GRIP AND DEODORIZER

(71) Applicant: Marc Alexander HORTON, Keswick (CA)

(72) Inventor: Marc Alexander HORTON, Keswick (CA)

(21) Appl. No.: 16/997,423

(22) Filed: Aug. 19, 2020

(51) Int. Cl. A47L 23/28; B65D 83/38; B65D 83/68; A61L 2/22; B05B 11/00

(52) U.S. Cl. CPC A47L 23/28; B65D 83/38; B05B 11/3085; A61L 2/22; B65D 83/68

(57) ABSTRACT

The invention disclosed herein relates a device that contains two separate and distinct aqueous formulations. One of the formulations has deodorizing properties and the other formulation has grip enhancing properties. The device has a means of applying the deodorizing formulation to the inner surfaces of shoes, typically athletic shoes, and a means of applying the grip enhancing formulation to the soles of the shoes. The invention also includes methods of deodorizing athletic shoes and methods for increasing shoe grip on a court surface.

### FIELD OF THE INVENTION

The present disclosure relates to methods and formulations for application to shoes, particularly to athletic shoes, more particularly to formulations that improve the grip of athletic shoes to playing surfaces, and alternative formulations that deodorize athletic shoes.

### BACKGROUND

Today's athletic shoes are expensive to purchase. They can look almost brand new after 6-12 months of wear, however, they frequently lose their intended effectiveness. They may dry out and become hard and slick as they collect dirt and debris, and are commonly replaced because of the lack of grip they have on a playing surface and the foot odor that develops within the shoe.

All athletes know the importance of keeping the soles of their shoes clean, as wax or dirt buildup on rubbery soles means less grip and more slips. Performance suffers as athletes lose control and traction. Numerous products are available that can either clean or increase the tack or grip of the sole of a shoe. However, these products are often sticky and made of toxic, corrosive, or non-environmentally friendly components and some just mask the problem. Some of the products are fabricated as a tack or traction mat. Dust, dirt and wax buildup can be removed from athletic shoe soles by stepping on the mat. Similar mats are available to impart a tacky layer to the sole of athletic shoes when stepping off. These products work well, but they are bulky to transport and are not always available in convenient locations for athletes. Other products are available in portable containers consisting of aerosol sprays which leave a tacky layer upon which anti-slip particles can be applied. Not only do the propellants in these aerosols damage the environment, but the anti-slip particles can damage the playing surface.

Another issue that affects the longevity of athletic shoes is foot odor. Again, there are numerous remedies available ranging from moisture wicking socks, to carbon-based odor absorbers to perfumed sprays that mask odor. There are also many home remedies, including inserting kitty litter in perforated containers inside the shoes and storing shoes in a freezer. Many athletes have two of more pairs of shoes, using them alternatively, thereby allowing the shoes to dry completely between each wearing. Most of these remedies do not address the root cause of the lingering odor associated with athletic footwear, which is the build-up of bacteria.

Numerous different containers are known that are capable of dispensing one or more similar or different liquids. The liquids may be dispensed manually using pump-action nozzles or from pressurized containers using various propellants known in the field. For example:

CN205170730 discloses a spraying instrument used for cleaning sports shoes. A hydrophobic thin oil layer is applied to the shoe to provide a self-cleaning function. The instrument also can prevent mold and bacteria growing in the shoe by applying a solution that has a deodorant effect.

US20130224388 discloses a pressurized spray can for applying adhesive and anti-slip particles to sports devices. The spray can comprise a propellant, at least one adhesive and anti-slip particles. In a one-part design, the anti-slip particles are added directly to the adhesive. In a two-part design, the anti-slip particles are stored in a second chamber of the spray can and can be added to the adhesive.

U.S. Pat. No. 6,196,421 discloses an aerosol can having two terminal portions each with its own valve and product delivery head of various configurations. The can may feature either one or two products for delivery, and can employ one or two volumes of propellant. The same material can also be distributed in two different manners, such as fine mist or strong stream, the mode of delivery being spray head dependent.

U.S. D846995, U.S. D817633, and U.S. D810581 illustrate designs for dual chambered fluid dispensers, each with different interior configurations for containment of liquid.

EP2377778 discloses a multiple spray can having at least two pressure vessels, which can be connected to one another either on the front side at their bottom walls or laterally to form a spray can arrangement.

U.S. Pat. No. 5,803,268 discloses a double-ended bottle for perfume having two separate compartments. Each of two interior spaces has its own closure/dispenser (e.g. spray fitting or pump), one at each end, so that two different liquids can be contained and dispensed separately in different ways.

U.S. Pat. No. 7,770,755 discloses a device for packaging and dispensing several fluid products. The device includes at least two sealed flexible bags arranged in a single rigid container, associated with at least one pump, the rigid container consisting of an open cylinder and the bags are provided each with a pump and a push button, and are mounted opposite on each of the two opposite openings of the cylinder. The device is applicable to appliances containing fluids to be dispensed independently of one another.

GB2281895 discloses a receptacle of elongate form defining, within its interior, two chambers to contain different commodities. The two chambers are each openable separately to dispense the contents of each chamber.

None of the above cited documents, alone or in combination satisfy the need for a solution to problem of athletic shoe odor and loss of grip. There therefore remains a need for a convenient, inexpensive and portable combined solution to tackle both of these problems.

### BRIEF SUMMARY

It is an object of the invention to provide formulations and methods for athletic shoe grip and deodorizer.

In accordance with an aspect of the invention there is provided a device containing two different formulations for improving the condition of athletic shoes, said device comprising: a container having first and second compartments containing first and second formulations, said first compartment having a means of applying said first formulation to an inner surface of said shoe, and said second compartment having a means of applying said second formulation to a sole of said shoe; the first formulation, contained within said first compartment, is an aqueous solution having deodorizing properties; and the second formulation, contained within said second compartment, is an aqueous solution having grip enhancing properties.

In accordance with another aspect of the invention there is provided a method of extending the life of an athletic shoe, comprising the steps: spraying a first formulation comprising: water; acid or acids; oil or oils; and inorganic or organic compound or compounds, onto the inner surfaces of said athletic shoe; and spraying a second formulation comprising: water; sugar, urethane pre-polymers; and aromatic diisocyanates, onto the sole of said athletic shoe.

In accordance with another aspect of the invention there is provided the use of a device containing first and second formulations to extend the life of an athletic shoe, said use comprising: spraying a first formulation comprising: water; vinegar; sodium bicarbonate; and one or more essential oils, onto the inner surfaces of said athletic shoe; and spraying the second formulation comprising: water; sugar, urethane pre-polymers; polymeric diphenylmethane-diisocyanate; and diphenylmethane diisocyanate, onto the sole of said athletic shoe.

The advantages and features of the present invention will become better understood with reference to the following more detailed description and claims taken in conjunction with the accompanying drawings in which like elements are identified with like symbols.

To easily identify the discussion of any particular element or act, the most significant digit or digits in a reference number refer to the figure number in which that element is first introduced.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an item 100 in accordance with one embodiment of the invention.

FIG. 2 illustrates an aspect of the subject matter in accordance with an embodiment.

FIG. 3 illustrates an aspect of the subject matter in accordance with another embodiment.

FIG. 4 illustrates an aspect of the subject matter in accordance with yet another embodiment.

### DETAILED DESCRIPTION

Devices and methods for carrying out the invention are presented in terms of embodiments depicted within the FIGS. However, the invention is not limited to the described embodiments, and a person skilled in the art will appreciate that many other embodiments of the invention are possible without deviating from the basic concept of the invention, and that any such work around will also fall under scope of this invention. It is envisioned that other styles and configurations of the present invention can be easily incorporated into the teachings of the present invention, and the configurations shall be shown and described for purposes of clarity and disclosure and not by way of limitation of scope.

The present invention describes a container that is capable of dispensing two different formulations, typically via a pump action spray nozzle. The dual compartment, dual ended liquid spray can be utilized for many products. In one embodiment of the invention, one of the formulations is a shoe deodorizer, powerful enough to fight strong foot odor, and the other formulation is a shoe grip solution, unique to the sport surface that the shoe is being used for.

Each foot typically has between 100,000 to 125,000 sweat glands which can secrete in the region of 125 ml of sweat a day. As feet are covered in shoes for most of the day, the moisture is sealed in which creates optimal breeding conditions for bacteria. As bacteria proliferate, they release unpleasant-smelling toxins, which are absorbed by shoes. Ridding shoes of odor is preliminary involved with killing the bacteria and subsequently creating an environment that is not conducive to their continued growth, but after the bacteria have been killed, it is also necessary to remove the odor that they have left behind.

It is contemplated that various different embodiments of the liquid deodorizing formulations can be used to kill bacteria, and/or prevent further growth and/or remove odor from shoes. Different embodiments may comprise components including, but not limited to, vinegar, alcohol, sodium bicarbonate and essential oils.

White vinegar is a disinfectant that is great for tackling bacteria. Spraying a mixture of white vinegar and distilled water into each shoe lining and inner sole, should kill most strains of bacteria. It is then important to allow the shoes to air dry before wearing them next.

Compounds such as sodium bicarbonate can neutralize bacterial acid produced by Brevibacteria as they feed off the sweat deposited inside shoes. Studies have shown that show that one gram of sodium bicarbonate can neutralize 12 milli-equivalents of malodorous bacterial acid.

Some formulations may also contain rubbing alcohol, which is an antiseptic that evaporates quickly and its drying effect will force moisture to evaporate along with it.

Some formulations may additionally contain fragrance or essential oils to impart a clean and fresh aroma to the spray. Essential oils are organic compounds extracted from plants, many of which have therapeutic properties. Fragrance (or “perfume”) oils are synthetic and they duplicate the aroma that essential oils have, but they do not exhibit any of the therapeutic properties. There are hundreds of essential oils that are reported to have therapeutic benefits in many different areas of medicine, from anxiety and depression to arthritis and allergies. Hence they are widely used, topically or systemically, to fight bacterial infection. They can also be used topically as antifungals, finding applications in the treatment of athletes' foot and toe nail fungus. Evidence shows that essential oils can effectively kill bacteria, without creating antibiotic resistant strains, making them great antibacterial and antifungal resources.

Embodiments of the invention may contain one of more essential oils selected from: anise; basil; bergamot; birch; black pepper, camphor; carrot seed, cedarwood; cinnamon; citronella; clary sage, clove, chamomile, coriander, cypress, eucalyptus, fennel, frankincense; galbanum; geranium; ginger; grapefruit; helichrysum; hysslop; jasmine; juniper, lavender; lemon; lemongrass; lime, mandarin, marjoram, melissa, myrrh; neroli; niaouli; orange; oragano; palma rosa; parsley; patchouli; peppermint; petitgrain; ravensara; rose; rosemary; sage; sandalwood; spearmint; spikenard; spruce; tangerine; tea tree, thyme; vanilla; vetiver; wintergreen; yarrow; and ylang ylang.

Preferable embodiments likely contain one or more of the following essential oils having proven antibacterial and antifungal properties: basil, bergamot, camphor; cedarwood, cinnamon, clove, coriander, eucalyptus, geranium, orange, lavender, lemon, lemon grass, lime; Oregano, peppermint, perilla rosemary, rosewood, tea tree, thyme.

Table 1 is taken from: Journal of Antimicrobial Chemotherapy, Volume 47, Issue 5, May 2001, Pages 565-573, Shigeharu Inouye, Toshio Takizawa, Hideyo Yamaguchi. This Table shows the efficacy of various essential oils in combating various bacterial infections.

TABLE 1

| | | | S. pneumoniae | S. pneumoniae | | |
|---|---|---|---|---|---|---|
| Essential oil | H. influenzae | S. pyogenes | IP 692 | PRC 53 | S. aureus | E. coli |
| Cinnamon bark | 3.13 | 6.25 | 3.13 | 1.56 | 6.25 | 12.5 |
| oil | | | | | | |
| Lemongrass oil | 1.56 | 6.25 | 6.25 | 6.25 | 12.5 | 100 |
| Perilla oil | 12.5 | 12.5 | 12.5 | 25 | 50 | >800 |
| Thyme (wild) oil | 3.13 | 6.25 | 3.13 | 3.13 | 12.5 | 12.5 |
| Thyme (red) oil | 3.13 | 3.13 | 6.25 | 6.25 | 6.25 | 12.5 |
| Thyme (geraniol) | 12.5 | 12.5 | 6.25 | 6.25 | 50 | >1600 |
| oil | | | | | | |
| Peppermint oil | 12.5 | 25 | 25 | 25 | 25 | >1600 |
| Tea tree oil | 25 | 50 | 50 | 50 | 50 | 50 |
| Coriander oil | 12.5 | 25 | 25 | 25 | 50 | 50 |
| Lavender (spike) | 12.5 | 25 | 25 | 25 | 50 | >1600 |
| oil | | | | | | |
| Lavender (true) | 25 | 50 | 50 | 50 | 100 | >1600 |
| oil | | | | | | |
| Rosemary oil | 50 | 50 | 50 | 50 | 100 | >1600 |
| Eucalyptus | 25 | 50 | 50 | 100 | 200 | >1600 |
| (radiata) oil | | | | | | |
| Citron oil | 200 | 200 | 400 | 400 | 800 | |

In one study, the essential oils of aegle, ageratum, citronella, eucalyptus, geranium, lemongrass, orange, palmarosa, patchouli and peppermint, were tested for antibacterial activity against 22 bacteria, including Gram-positive cocci and rods and Gram-negative rods, and twelve fungi. Lemongrass, eucalyptus, peppermint and orange oils were effective against all the 22 bacterial strains. Aegle and palmarosa oils inhibited 21 bacteria; patchouli and ageratum oils inhibited 20 bacteria and citronella and geranium oils were inhibitory to 15 and 12 bacterial strains, respectively. All twelve fungi were inhibited by seven oils (aegle, citronella, geranium, lemongrass, orange, palmarosa and patchouli). Eucalyptus and peppermint oils were effective against eleven fungi. Ageratum oil was inhibitory to only four fungi tested. Microbios. 1996; 86(349):237-46. Pattnaik et al.

It is also contemplated that embodiments of the invention may contain one or more fragrance oils that will impart a scent to the deodorizing formulations, typically in place of one or more essential oils. These embodiments are intended to mask the odor produced by the bacterial waste products within the lining of the athletic shoe, as opposed to functioning as antibacterial agents.

The following formulations serve as examples of different combinations of various reagents that can be used as an anti-odor solution. It will be understood by workers skilled in the art that numerous combinations of reagents including, but not limited to: alcohols (such as methanol, ethanol, propanol, and butanol); acids (such as vinegar, white vinegar, apple cider vinegar, lemon juice, and lime juice); oils (such as castor oil, essential oils and fragrance oils); inorganic compounds (such as sodium bicarbonate, calcium phosphate, and magnesium sulphate); and organic compounds (such as mono or polypropylene glycol, zinc ricinoleate, o-phenylphenol, 4-tert-amlyphenol, glycerine, triclosan, benzylkonium chloride, kathon, propenamide and propenic or propionic acid); can be mixed in varying combinations and quantities to produce an anti-odor solution that can be used in the embodiments of the invention as described herein.

### Deodorizing Formulation 1:

Distilled (or deionized) water (75%-90%)

Vinegar (8%-15%)

Essential oil or oils (0.1-2.0%)

Sodium bicarbonate (3%-10%)

In deodorizing formulation 1, the combination of vinegar and sodium bicarbonate produce a solution containing active ingredients carbonic acid and sodium acetate. Preferred essential oils include, but are not limited to oils selected from the group consisting of lemongrass, orange and lavender.

### Deodorizing Formulation 2:

Distilled (or deionized) water (75%-90%)

Apple cider vinegar (8%-15%)

Essential oils selected from: tea tree; peppermint; grapefruit; bergamot; clove; thyme, and eucalyptus oils (total 0.5-2%)

Sodium bicarbonate—(3%-10%)—optional

### Deodorizing Formulation 3:

Distilled (or deionized) water (75%-90%)

Essential oils selected from: tea tree; peppermint; chamomile (total 0.5-2%)

Witch hazel (up to 10%)

Aloe vera (1.0-5.0%)

Vitamin E (0.1-0.5%)

Olive extract (1.0-5.0%)

### Deodorizing Formulation 4:

Deionized water (70-80%)

Isopropyl alcohol (5-15%)

Monopropylene glycol (1-3%)

Glycerine (1-3%)

Hydrogenated castor oil (1-5%)

Peppermint oil (0.1-1.0%)

Eucalyptus oil (0.1-1.0%)

Thyme oil (0.1-1.0%)

Triclosan (0.1-0.5%)

Benzylkonium chloride (0.1-0.3%)

Kathon (0.1-0.3%)

Mix hydrogenated castor oil and essential oils. Dissolve glycol, glycerine in water and add alcohol. Combine these solutions and slowly add remaining constituents and mix until resulting solution is homogenous.

### Deodorizing Formulation 5:

Distilled water (75%-90%)

Sodium bicarbonate (3%-10%)

Magnesium sulphate (3%-10%)

2-Propenamide (2%-5%)

2-Propenic acid (sodium salt) (2%-5%)

Tricalcium phosphate (2%-5%)

Benzethonium chloride (0.1-0.3%)

Scent or fragrance oils or essential oils (0.1-2.0%)

Numerous different formulations are contemplated in regard to the grip enhancing solutions that function to increase the grip the soles of the athletic shoes have to the playing surface. Different embodiments may comprise components including, but not limited to, water (distilled, deionized, sterilized, and/or reverse osmosis), sugar (glucose, fructose, sucrose, lactose, galactose, and/or maltose), aluminum chloride hexahydrate, vinyl acetate, polyvinyl acetate, urethane prepolymer, diphenylmethane-diisocyanate, and isomers, and homologues thereof.

It is desirable that grip compositions having prolonged tackiness and bonding properties can be applied to the soles of athletic shoes over a wide range of temperatures i.e. at least −20° C. to 40° C. Typically the grip solution is applied prior to each game or scrimmage for maximum grip. There will remain some level of increased grip even when the grip formulation is not reapplied for every game or scrimmage. The grip compositions are formulated to provide a certain minimum balance of physical and chemical properties so as to be able to withstand the stresses and environment that the product will encounter and be expected to survive in use. The physical properties known in the art as tack, peel adhesion, and shear adhesion (otherwise referred to as holding power) are important in determining the suitability of any given grip composition to provide the necessary application properties and in-service durability required of the grip product.

The first property, tack, is in effect the instantaneous adhesion of the grip layer to the surface of the sole of the shoe immediately after contacting the surface with the layer. One accepted method of measuring tack, endorsed by the Pressure Sensitive Tape Council (USA), is test method PSTC-6 evolved and published as a standard test by the Council. It involves rolling a stainless steel ball down an inclined plane of the surface after a layer of the grip composition is deposited, and measuring the distance the ball rolls. In this test, tack is expressed in distance of ball travel. Thus high tack adhesive stops the ball within 0.5 to 3 inches (about 1.27 to 7.6 cm), whereas low tack adhesives allow the ball to roll more than 12 inches (about 30.3 cm).

The second property, peel adhesion, is the adhesion of the grip product after the adhesive layer has been in contact with an adherent surface, such as a basketball court, for a sufficient period of time to reach a steady state or equilibrium with the surface. It essentially corresponds the force required to remove an grip-treated athletic shoe from court surface at a specified angle and speed. PSTC-1 specifies one standard test method.

The third physical property utilized to define the suitability of a grip product for the desired use is its shear adhesion. This test measures in essence the durability and permanence of the adhesive bond to a shoe surface when subjected to a constant lateral force. PSTC-7 specifies one standard test method.

Besides these physical property parameters, the chemical attributes of the grip composition must also be considered in order to provide a grip product having the requisite durability in the environment in which it is to be used. Thus, for example, the grip composition must be formulated so as to exhibit resistance to degrading environmental conditions such as elevated temperatures, sunlight, organic solvents or cleaning products used on court surfaces, etc.

Various polymeric materials have been used as a basis for the manufacture of pressure-sensitive adhesives and grip or tack materials, including natural rubber, styrene butadiene copolymers and acrylate copolymers. However, the preferred components of the grip composition of embodiments of the invention disclosed herein relate to polyurethane based grip solutions made from commonly available urethane raw materials, and optionally tackifying resins or plasticizers. The polyurethanes disclosed are made from aromatic diisocyanates and diols of hydroxyl number between about 56 and about 265 and triols of hydroxyl number between about 28 and about 56.

Aromatic diisocyanates are of particular effective in increasing the tack of surfaces to which they are applied. Specifically, diphenylmethane-diisocyanate, or methylene diphenyl diisocyanate (MDI), and isomers and homologues thereof. MDI has three common isomers which are formed by attaching the isocyanate groups at different positions around the rings: 2,2′-MDI, 2,4′-MDI, and 4,4′-MDI. The 4,4′ isomer is most widely used in the adhesives industry, and is also known as 4,4′-diphenylmethane diisocyanate. The 4,4′-MDI is therefore particularly useful in the formulations intended to improve grip of athletic shoes to playing surfaces. Importantly, MDI has a relatively low human toxicity.

Polymeric MDI (PMDI) is another component to the grip formulation. It is the primary commercial form of MDI, is actually a mixture that contains 25-80% monomeric 4,4′-MDI as well as oligomers containing 3-6 rings and other minor isomers, such as the 2,2′-isomer and the 2,4′ isomer. The exact composition of PMDI varies with the manufacturer.

An additional component of an embodiment of the grip formulation is urethane or polyurethane prepolymer. Urethane/polyurethane prepolymers are formed by combining an excess of diisocyanate with a polyol. The prepolymers play a vital role in polyurethane chemistry and are used in many applications, including the production of adhesives, sealants, and binders.

Vinyl acetate polymer is a reagent in some of the grip formulations. The vinyl acetate monomer can be polymerized while dispersed in water to form a milky-white emulsion. This fluid can be processed directly into flexible, adherent films.

Aluminum chloride hexahydrate is another reagent that may be used in the preparation of the second formulation t is used in various chemical applications as a Lewis base,

The grip formulation also contains mono and disaccharides such as glucose, fructose, sucrose, lactose, galactose, and maltose.

### Grip Formulation 1:

Distilled water (60%-80%)

Sugar, typically sucrose (5-10%)

Urethane pre-polymer (10%-20%)

Polymeric Diphenylmethane-diisocyanate (2%-10%)

Diphenylmethane diisocyanate, mixed isomers—but predominantly 4,4′MDI (1%-2%)

Diphenylmethane diisocyanate homologues (1%-10%)

### Grip Formulation 2:

Distilled Water 70%-90%

Sugar, typically sucrose 5%-10%

Vinyl acetate polymer 5%-15%

Aluminum chloride hexahydrate—less than 1%

Grip formulations 1 and 2 are made each made in two stages: mixing and heating the reagents and then adding the sucrose while stirring.

With regards to the method of application of the formulations to the athletic shoes, this will be better understood by reference to the figures.

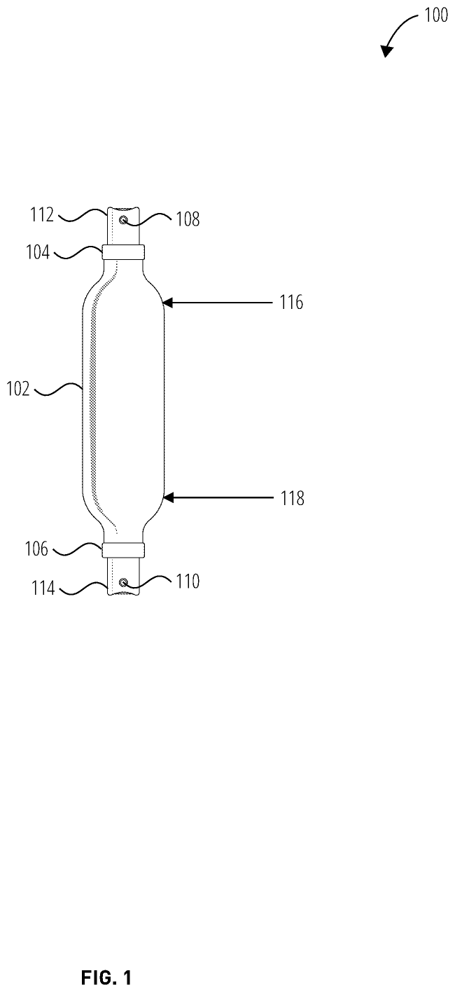

FIG. 1 illustrates a side view of an embodiment of the application container 102. The container 102 has a first end 116 having a first dispenser 112 attached to it by a means of attachment 104. The first dispenser 112 is equipped with a first spray nozzle 108. Additionally, the container 102 has a second end 118 having a second dispenser 114 attached to it by a means of attachment 106. The second dispenser 114 is equipped with a second spray nozzle 110.

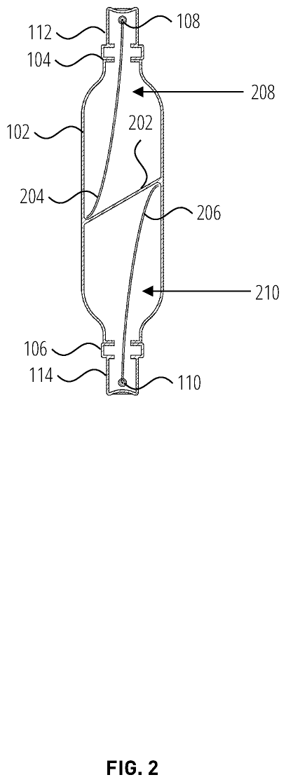

FIG. 2 illustrates a cross-sectional view of an embodiment of the application container 102. The relative positions of the first dispenser 112 and second dispenser 114 can be seen at opposite ends of the container 102. This figure clearly shows the first end 116 houses a first compartment 208 and the second end 118 houses a second compartment 210. The first spray nozzle 108 is connected to the liquid (not shown) in the first compartment 208 by a first tube 204. Similarly, the second spray nozzle 110 is connected to the liquid (not shown) in the second compartment 210 by a second tube 206.

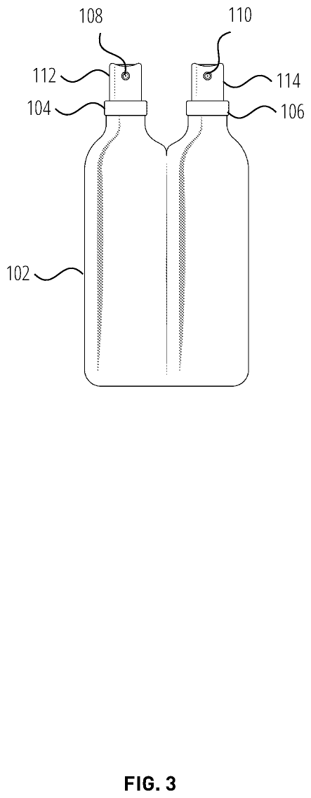

FIG. 3 illustrates a side view of an additional embodiment of the application container 102 of the present invention. In this embodiment both the first dispenser 112 and second dispenser 114 are situated at the first end 116 of the container 102.

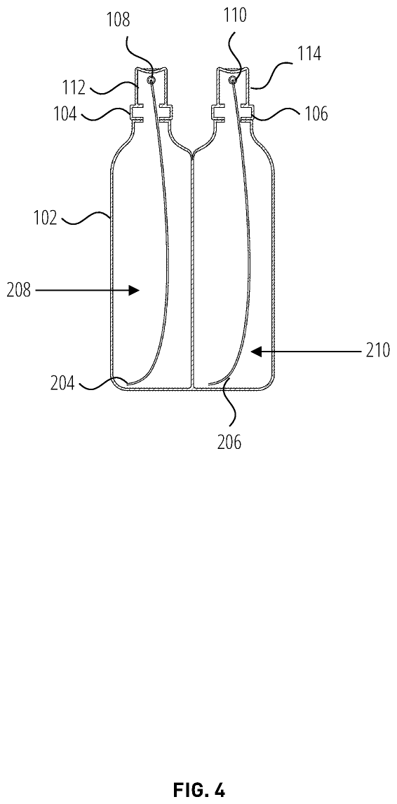

FIG. 4 illustrates a cross-sectional view of an embodiment of the application container 102. The relative positions of the first dispenser 112 and second dispenser 114 can be seen at the same end of the container 102. This figure clearly shows the position of the first compartment 208 and the second compartment 210. The first spray nozzle 108 is connected to the liquid (not shown) in the first compartment 208 by a first tube 204. Similarly, the second spray nozzle 110 is connected to the liquid (not shown) in the second compartment 210 by a second tube 206.

In the embodiments of the invention illustrated herein, the first compartment 208 and second compartment 210 are essentially configured to hold similar volumes of liquid. It is likely that most athletes will use the deodorizing formulation and grip formulation at a similar rate. However, it is contemplated that under certain conditions more of one formulation will be used more rapidly than the other. For example, in summer months, more sports may be played by recreational players on outdoor hard surfaces that would not require use of the grip formulation, and more of the deodorizer formulation would be required. Alternatively, some athletes who predominantly compete on wooden surfaces, and who do not have particularly sweaty feet, may require more of the grip formulation than the deodorizing formulation.

It is intended that the grip formulations are predominantly used for sports played on hardwood or wax floors including gymnasium floors or indoor track surfaces.

Typical sports that can benefit from athletic shoes having improved grip and traction are many and include, but are not limited to: basketball, volleyball, handball, racquetball, dodgeball, indoor soccer, Futsol, floorball/Unihockey, squash, badminton, tennis, table tennis, aerobics, fencing, indoor track events and fitness training and fencing.

In the first instance, it is intended that the two in one product is especially designed to help basketball players keep the grip on the bottom of their shoes, ensure their shoes do not have a lingering smell, and help extend the lifespan of expensive basketball shoes, by 4-12 months. Instead of purchasing a new pair of shoes or buying two different general (not always specific) products for grip and smell, it can solve these problems in one dual-action product in a much more cost-efficient manner.

Methods of enhancing the gripping performance and extend the life of all kinds of sports shoes within the scope of protection sought. The methods of the invention comprise spraying the formulation contained in the first compartment 208 of the container 102 onto the inner surfaces of the athletic shoe to reduce odor, typically just after use. The second step of the method involves spraying the formulation contained in the second compartment 210 of the container 102 onto the bottom or sole of the athletic shoe prior to playing to increase grip of the sole to the playing surface.

Use of the dual-purpose product should alleviate problems associated with foot odor and loss of grip, and prevent players having to purchase new basketball shoes every 6 months, an expensive endeavor not only for recreational and professional players, but also for referees.

Athletes are always trying to get an edge, and any product that will allow them to gain an advantage and extend the life of their equipment will be an important addition to their equipment bag. There is a certain stigma associated with carrying around a bottle of Febreeze™, it is contemplated that athletes will be comfortable using a custom made and specifically formulated product designed and engineered for their specific sport.

With numerous sports, including basketball, on the rise, the market conditions are set up right for a product like this. Especially when considering (even more during and likely also after COVID-19) that basketball players are playing on outdoor courts and losing grip faster than if they were playing in a school gym. Other considerations include various gym facilities and basketball courts, including LA Fitness basketball courts, that are becoming more and more popular. However, the gyms and LA Fitness' now becoming older and older, their courts and playing surfaces are slowly declining, and the grip on the floor is declining with it. More and more players will be looking to solve this problem. This product is easily engineered to other surfaces, courts and track used in sports.

The foregoing descriptions of specific embodiments of the present invention have been presented for purposes of illustration and description. They are not intended to be exhaustive or to limit the invention and method of use to the precise forms disclosed. Many modifications and variations are possible in light of the above teaching. The embodiments described were chosen and described in order to best explain the principles of the invention and its practical application, and to thereby enable others skilled in the art to best utilize the invention and various embodiments with various modifications as are suited to the particular use contemplated. It is understood that various omissions or substitutions of equivalents are contemplated as circumstance may suggest or render expedient, but is intended to cover the application or implementation without departing from the spirit or scope of the claims of the present invention.

1. A device containing two different formulations for improving the condition of athletic shoes, said device comprising:
a container having first and second compartments and first and second ends;
a first formulation having deodorizing properties, contained within said first compartment;
a second formulation, contained within said second compartment, having grip enhancing properties;
first spray nozzle positioned at said first end of said container for applying said first formulation to an inner surface of said shoes, and
second spray nozzle positioned at said second end of said container for applying said second formulation to a sole of said shoes.

2. The device of claim 1, wherein the first formulation comprises components selected from the group consisting of: water; acid or acids; oil or oils; and inorganic compound or compounds.

3. The device of claim 2, wherein the water is selected from the group consisting of: distilled; deionized; sterilized; and reverse osmosis, the acid or acids are selected from the group consisting of: vinegar; white vinegar; apple cider vinegar; lemon juice; and lime juice, the oil or oils are selected from the group consisting of: castor oil; fragrance oils; and essential oils; and the inorganic compound or compounds are selected from the group consisting of: sodium bicarbonate, calcium phosphate, and magnesium sulphate.

4. (canceled)

5. The device of claim 2, wherein the water is selected from the group consisting of: distilled; deionized; sterilized; and reverse osmosis, the acid or acids are selected from the group consisting of: vinegar; white vinegar; apple cider vinegar; lemon juice; and lime juice, the oil or oils are selected from the group consisting of: castor oil; fragrance oils; and essential oils; and the organic compound or compounds are selected from the group consisting of: mono or polypropylene glycol, zinc ricinoleate, o-phenylphenol, 4-tert-amylphenol, glycerine, triclosan, benzylkonium chloride, kathon, propenamide and propenic or propionic acid.

6. The device of claim 1, wherein the first formulation comprises: distilled water: vinegar; sodium bicarbonate and essential oil or oils.

7. The device of claim 6, wherein the first formulation comprises: 75-90% distilled water; 8-15% vinegar; and 3-10% sodium bicarbonate and 0.1-2.0% essential oil or oils.

8. The device of claim 6, wherein the essential oil or oils are selected from the group consisting of: anise; basil; bergamot; birch; black pepper, camphor; carrot seed, cedarwood; cinnamon; citronella; clary sage, clove, chamomile, coriander, cypress, eucalyptus, fennel, frankincense; galbanum; geranium; ginger; grapefruit; helichrysum; hysslop; jasmine; juniper, lavender; lemon; lemongrass; lime, mandarin, marjoram, melissa, myrrh; neroli; niaouli; orange; oragano; palma rosa; parsley; patchouli; peppermint; perilla; petitgrain; ravensara; rose; rosemary; rosewood; sage; sandalwood; spearmint; spikenard; spruce; tangerine; tea tree, thyme; vanilla; vetiver; wintergreen; yarrow; and ylang ylang.

9. The device of claim 6, wherein the essential oil or oils are selected from the group consisting of: lavender; lemongrass; and orange.

10. The device of claim 1, wherein the first formulation additionally comprises an alcohol selected from the group consisting of: methanol, ethanol; propanol; and butanol.

11. The device of claim 1, wherein the second formulation comprises components selected from the group consisting of: water; sugar; urethane pre-polymers; and aromatic diisocyanates.

12. The device of claim 11, wherein the water is selected from the group consisting of: distilled; deionized; sterilized; and reverse osmosis, the sugar is selected from the group consisting of: glucose; fructose; sucrose; lactose; galactose; and maltose, and the aromatic diisocyanates are selected from the group consisting of: polymeric diphenylmethane-diisocyanate; diphenylmethane diisocyanate and isomers and homologues thereof.

13. The device of claim 12, wherein the second formulation comprises: 60-80% distilled water; 5-10% sucrose; 10-20% urethane pre-polymer; 2-10% polymeric diphenylmethane-diisocyanate; and 1-2% diphenylmethane diisocyanate and isomers or homologues thereof.

14. The device of claim 12, wherein the isomers of diphenylmethane diisocyanate (MDI) are selected from the group comprising: 2,2′-MDI, 2,4′-MDI, and 4,4′-MDI.

15. The device of claim 1, wherein the second formulation comprises components selected from the group consisting of: water; sugar, vinyl acetate polymer, aluminum chloride hexahydrate.

16. The device of claim 1, wherein the first formulation comprises: distilled water: vinegar; essential oil or oils; and sodium bicarbonate, and the second formulation comprises: distilled water; sugar; polymeric diphenylmethane-diisocyanate; diphenylmethane diisocyanate and isomers and homologues thereof

17. The device of claim 1, wherein the first formulation comprises: distilled water: vinegar; essential oil or oils; and sodium bicarbonate, and the second formulation comprises: distilled water; sugar; vinyl acetate polymer and aluminum chloride hexahydrate.

18. A method of extending the life of an athletic shoe, comprising the steps:
spraying a first formulation comprising: water; acid or acids; oil or oils; and inorganic or organic compound or compounds, onto the inner surfaces of said athletic shoe; and
spraying a second formulation comprising: water; sugar, urethane pre-polymers; and aromatic diisocyanates, onto the sole of said athletic shoe.

19. The method of claim 18, wherein the first formulation comprises: distilled water: vinegar; sodium bicarbonate and essential oil or oils.

20. The method of claim 18, wherein the second liquid comprises components selected from the group consisting of: water; sugar; urethane pre-polymers; and aromatic diisocyanates.
